(19) United States
(12) Patent Application Publication
(10) Pub. No.: US 2021/0338961 A1
(43) Pub. Date: Nov. 4, 2021

(54) Positive air pressure mask system

(71) Applicant: Jerry L. Godwin, Bel Air, MD (US)

(72) Inventor: Jerry L. Godwin, Bel Air, MD (US)

(73) Assignee: Jerry L. Godwin, Bel Air, MD (US)

(21) Appl. No.: 16/873,528

(22) Filed: Apr. 30, 2020

(51) Int. Cl. A61M 16/06

(52) U.S. Cl. CPC A61M 16/0683; A61M 16/0622; A61M 16/0666; A61M 16/0694

(57) ABSTRACT

A positive airway pressure mask system is described. The mask is comprised of a non-elastic or non-stretchable headgear, wherein the chin strap of the headgear prevents the jaw of the user from dropping open, thus limiting or preventing mouth breathing at night. The mask has attached or integral with it a nose pillow which has attached nostril feed pieces for directing air directly into the nostrils. This mask is adjustably connected to the non-stretchable headgear at specific locations, allowing for an unlimited number of adjustments to properly fit the mask to an individual's face, providing comfort and efficacy of use.

A positive airway pressure mask system is shown which is used by a patient when asleep to prevent or minimize air leakage while being comfortable.

### BRIEF DESCRIPTION OF THE DRAWINGS

FIG. 1 is a side view showing the headgear;

FIG. 2 is a perspective view of the headgear;

FIG. 3 is a first side perspective view of the positive airway pressure mask system including the points of attachment;

FIG. 4 is a second side perspective view of the positive airway pressure mask system including points of attachment;

FIG. 5 is a side view of another perspective view of the positive airway pressure mask system including of attachment:

FIG. 6 is an overhead view of the chin strap of the headgear;

FIG. 7 is a rear view of one embodiment of the crown strap section;

FIG. 8 is a bottom view of one embodiment of the crown strap section;

FIG. 9 is a rear view of the alternative embodiment of the straps;

FIG. 10 is a perspective view of the alternative embodiment of the straps attached to the pressure mask;

FIG. 11 is a perspective view of an embodiment of a nasal pillow;

FIG. 12 is a perspective yet another embodiment of the nasal pillow;

FIG. 13 is a perspective view of a nose mask;

FIG. 14 is a perspective view of another embodiment of a nose mask;

FIG. 15 is a perspective view of a full face mask attached to the headgear;

FIG. 16 is a perspective view of another embodiment of a full face mask attached to the headgear; and

FIG. 17 is a perspective view of a nasal pillow.

The figures depict various embodiments of the described device and are for purposes of illustration only. One skilled in the art will readily recognize from the following discussion that alternative embodiments of the methods and kits illustrated herein may be employed without departing from the principles of the methods and kits described herein.

### DETAILED DESCRIPTION OF THE DISCLOSURE

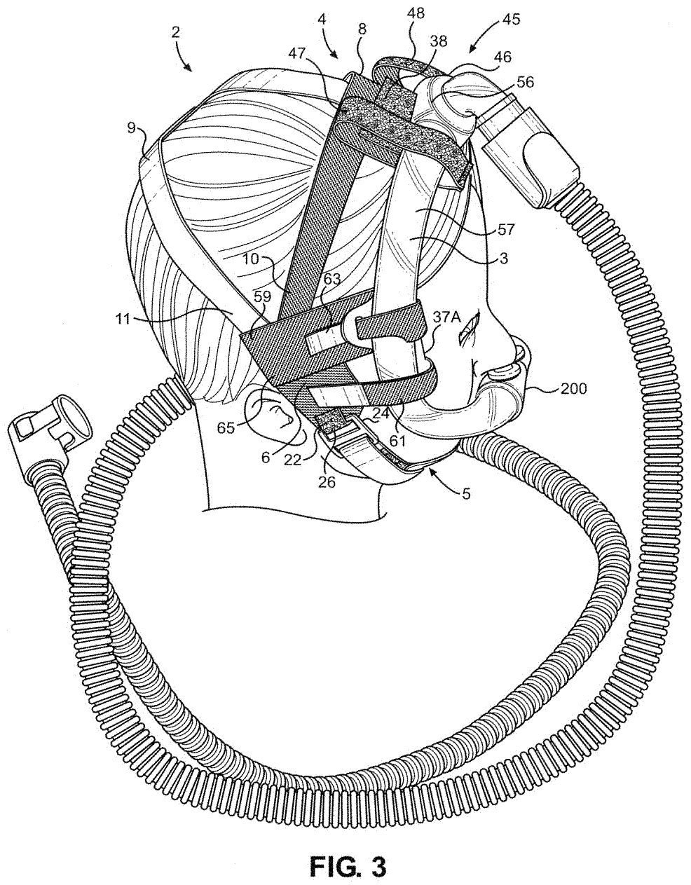

FIG. 3 shows the PAP mask system 1 comprising two parts, with the two parts comprising a headgear 2 and a mask 3. This PAP mask system is to be used to treat patients with sleep apnea. Thus, it is advantageous that the PAP mask system to be designed to minimize the mask air leakage and eliminate the mouth breathing air leakage thereby assuring the effectiveness of the PAP system, while retaining a comfortable attachment means.

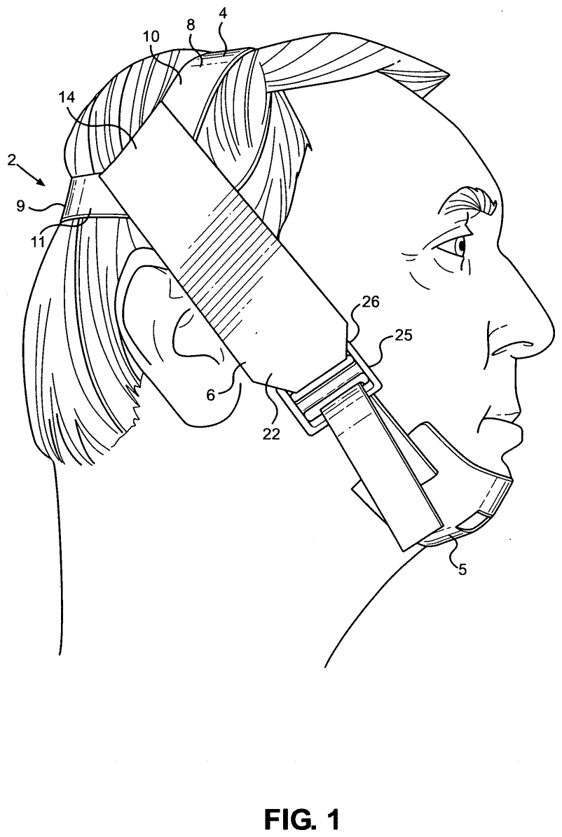

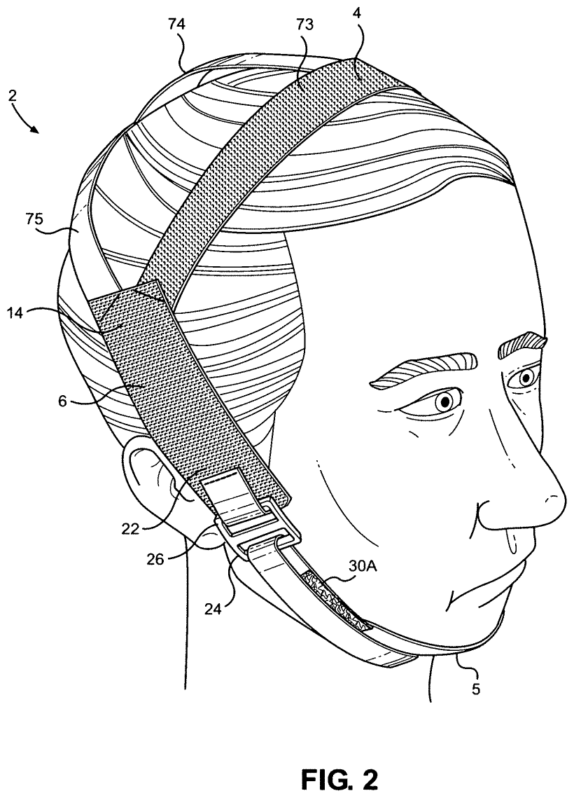

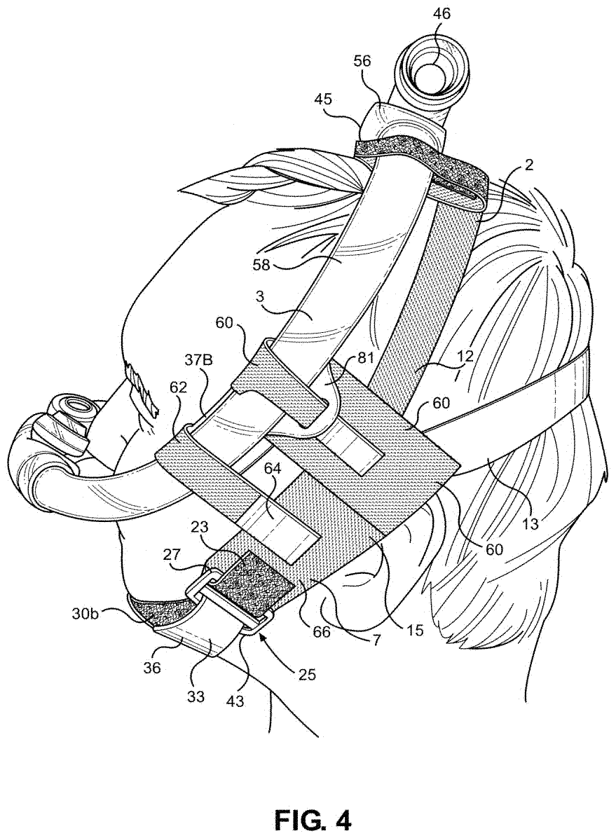

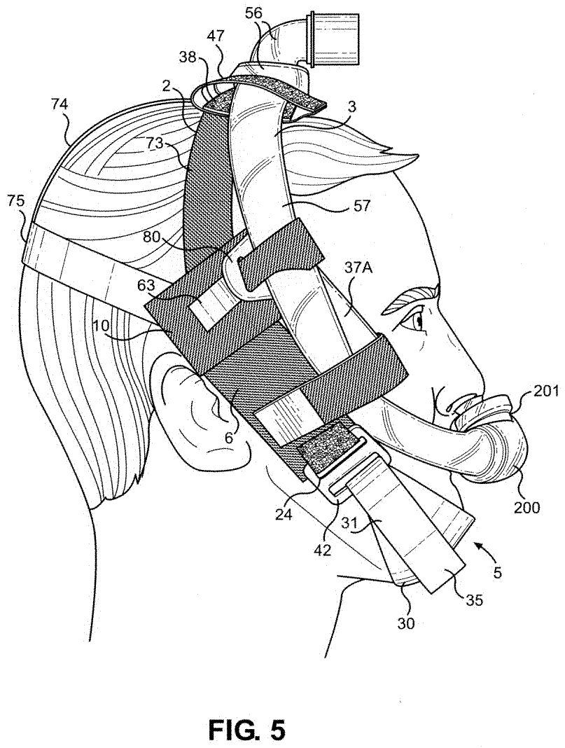

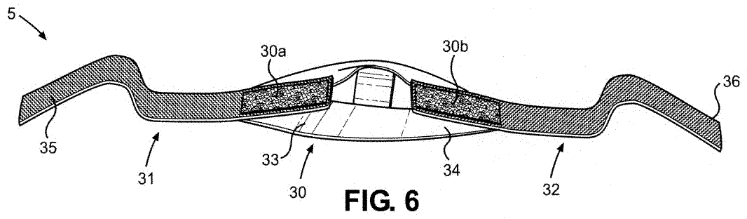

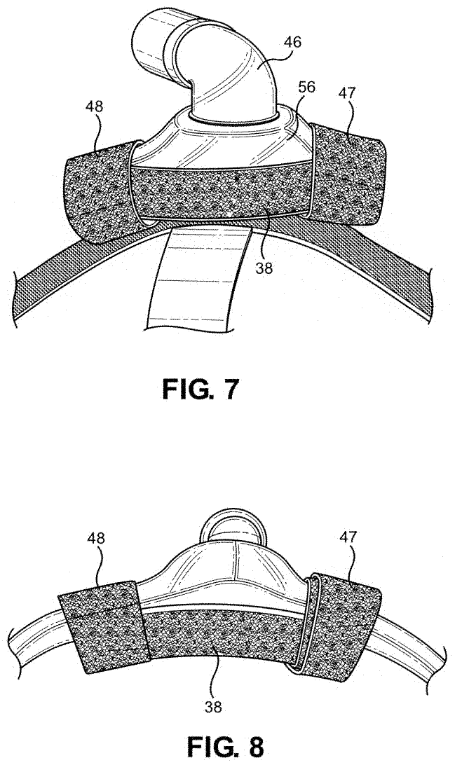

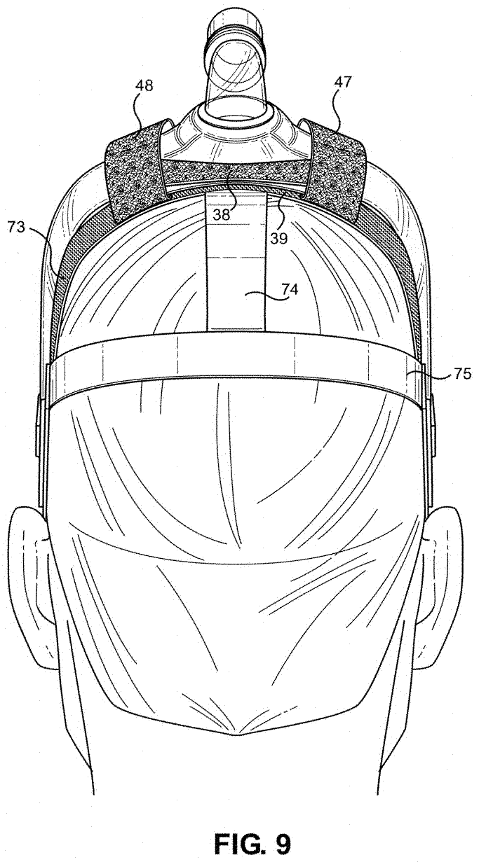

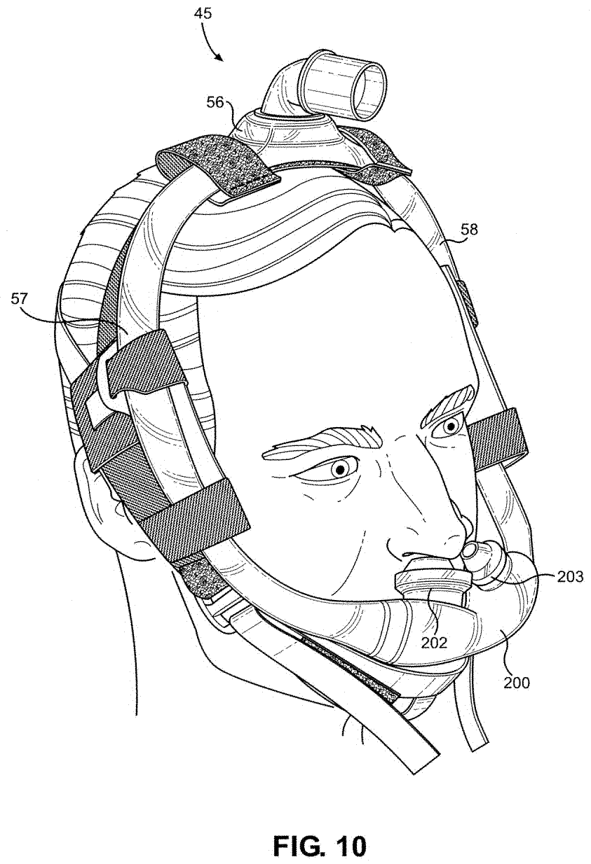

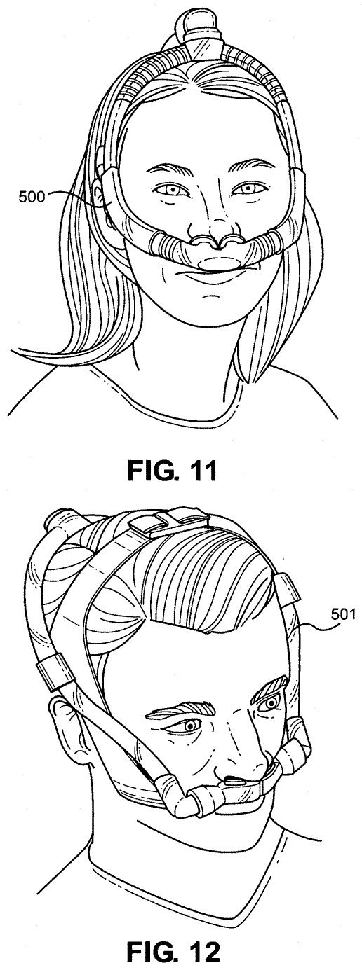

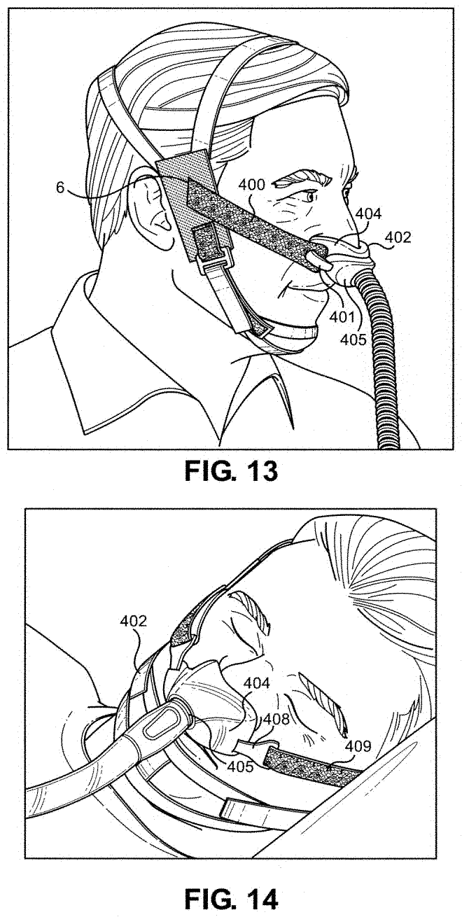

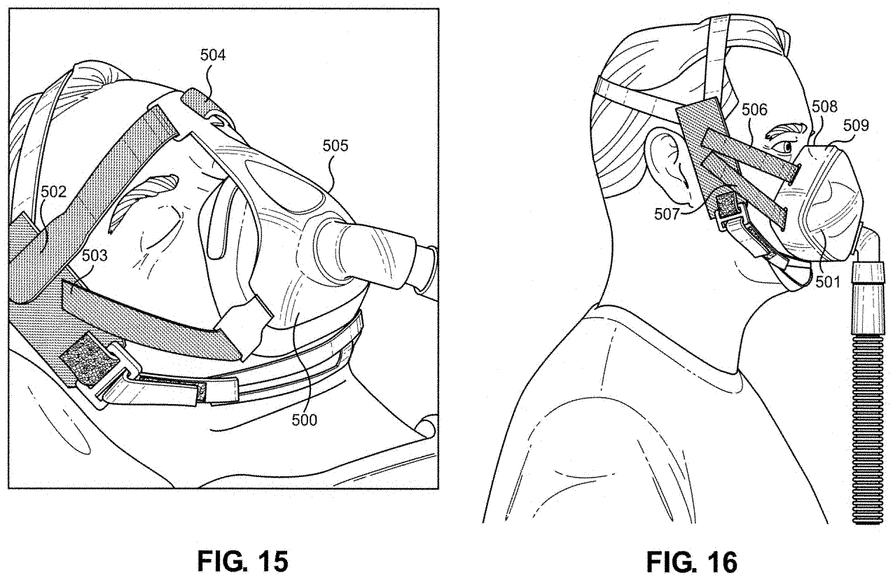

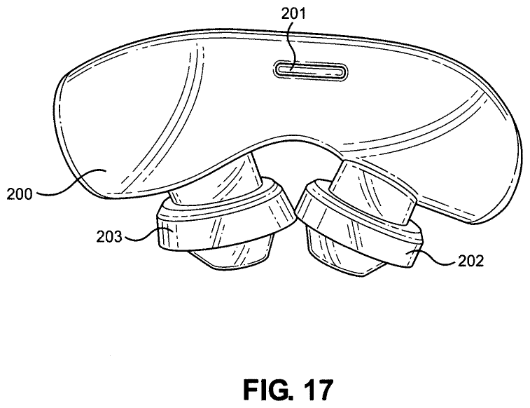

The headgear 2 (FIGS. 1, 2 and 4) is itself comprised of at least four sections, a crown strap section 4, a chin strap 5, and two headgear connector strips 6, 7. In this embodiment, the crown strap section 4 has at least one or two crown straps 8 (the first or lead crown strap), 9 that fit over the crown of the head. Each of the two crown straps 8, 9 have a first end 10, 11 and a second end 12, 13. The first end 10, 11 of each of the two crown straps 8,9, are sewn to the first end 14 of the headgear connector strip 6, and the second ends 12, 13 of crown straps 8,9 are sewn to the first end 15 of the second headgear connector strip 7. In another embodiment, the two ends of the two mated sections have male snaps and female snaps and are thus snapped together. In another embodiment, there is a plurality of female snaps and/or male snaps. In yet another embodiment, the two ends and are connected by at least one button and at least one button hole. In yet another embodiment, a hook and loop arrangement are used to connect the each of the two ends. Any permanent and non-permanent method to connect the ends may be used.

Each of the two headgear connector strips 6, 7 have a second end 22, 23 which in one embodiment has attached to a first end 26, 27 of a buckle 24, 25. In one embodiment, the second end 22, 23 of the headgear connector strips 6, 7 is looped around the one end 26, 27 of the buckle 24, 25 and sewn back on itself.

The chin strap 5 has three central pieces; the chin rest 30, a first strap 31, and a second strap 32. Both the first strap 31 and the second strap 32 have a first end 33, 34 removably attached or integral with the chin rest 30. In one embodiment, there is a first and second hook and loop strip 30a and 30b positioned on each end of the underside of the chin rest 30. In such an arrangement, the first chin strap 31 and second chin strap 32 are both made out of hook and loop structures. In such an arrangement, the first ends 33, 34 of chin straps 31, 32 are attached (by hook and loop, glue or by sewing) to the end or underside end of the chin rest 30. The seconds ends 35, 36 of chinstraps 32, 33 which fit through second end 42, 43 of the buckles 24, 25 and then removably attached to the respective hook and loop strips 30a 30b. The placement of chin straps 32, 33 depend on the size of the face of and the comfort factor of the user.

It should be noted that the distance between one's crown of the skull and nose always remains constant. Therefore, since the nasal pillow is positioned relative to the headgear and the headgear is secured to the skullcap which is located on the crown of the skull, the location of the nasal pillow relative to the nose never changes.

The headgear 2 is made of a sturdy but flexible non-elastic material with several sections having a hook and loop side. The material can be made of natural or artificial thread, or plastic. It should be noted that the term “non-elastic” refers to a material with negligible elasticity which is essential to the design and effectiveness of the mask system as well as comfort for the patient. Specifically, the purpose of the headgear 2 is to limit the amount of mouth breathing. The headgear 2 provides a sturdy foundation for the mask, but as stated earlier, also is designed to gently touch the skin. In one embodiment parts of the headgear are covered with a material (felt, for example) which will attach to a hook and loop material. In another embodiment, the material is strapping with no covering.

By eliminating the elasticity of the headgear 2, and due to the design of the headgear, there will be very limited movement of the chin rest 5, which limits the movement of and the opening of the mouth. In one embodiment, the mouth can move no more than about an eighth of an inch to about half of an inch, with the lips typically staying closed. In another embodiment, the mouth can move no more than about an eighth of an inch to about a quarter of an inch. The chin rest 5 attached to the rest of the headgear, prevents the jaw from dropping to the point where mouth breathing occurs, and therefore the patient receives maximum benefit from the PAP machine. The patient, when awake, is able to talk, cough, yawn, and breathe through the mouth with the mask and headgear installed due to the gentle fitting design.

It should be noted that in an alternative embodiment the crown strap section could have as few as one or as many as three or more straps, including at least one crown strap and other differently positioned head straps.

The headgear 2, is connected to the mask 3, a Philips Respironics DreamWear model, by means of additional hook and loop strips which will be explained, infra. The mask can be a nasal cushion (or pillow) mask, which covers the nostrils, or a nasal mask which covers the entire nose, or a full mask which covers the entire nose and mouth. The mask 3 could also be variations of all three of these masks. The mask 3 can be made of silicon or plastic, and can have a level of flexibility while still maintaining its overall shape while in use. The mask as shown is hollow, to let air flow through the mask to the nose pillow 200, which has two nostril feed pieces 202, 203 through which air flows. The mask 3 has four principal sections. The first section is the forehead section 45. In this embodiment there is an air passageway 56 through the mask. In this embodiment shown, a hose attachment 46 through which air first enters is positioned in the top side of center of the forehead section 45 of the mask 3. Wrapped around each side of the hose attachments/air passageways are hook and loop support structures or strips 47, 48, connected by a bridge 38 which can mate with the hook and loop strip 39 that is on top of or comprising the or integral with the crown strap section 4.

The second and third sections are the first and second side air passageway tubes 57, 58, which in this case are integral with the forehead section 45. These side air passageway tubes 57, 58 are connected to the headgear 2 by at least one hook and loop strap 59, 60 on each of the side air passageways tubes 57, 58, which as the name implies, allows air to pass from the hose attachment through the mask. In another embodiment the extensions at least two hook and loop straps 59, 61, 60, 62 on the passageway tubes 57, 58 connect to the hook & loop strips on the outer sides of headgear connector strips 6, 7. Specifically, the hook and loop straps 59, 61, 60, 62 are looped around the air passageway tubes and the hook and loop strap first ends 63, 64, can be connected to hook and loop strap second ends 65, 66, with hook and loop straps 59 and 61 connected by a left connector bridge 37a, and hook and loop straps, 60 and 62 connected by right connector bridge 37b on the inside wall of the mask, whereupon the connector bridges 37a, 37b can then connect to headgear connector strips 6, 7. In another embodiment, plastic loops 80, 81 are connected to or are integral with side passageway tubes 57, 58. In one embodiment, the loop 80, 81 allows the first ends 63, 64, which in this embodiment are narrowed, through loops 80,81 and then attached by hook and loop onto loop straps 59,60. In other embodiments the connection between the first ends and the loop strap can be by snaps or any other known method.

The fourth part of the headgear is the nasal feed piece 200, which allows for air through the passageway to be fed directly into the nose. The use of the hook and loop straps in combination with the headgear allows the nostril feed pieces 202, 203 to be brought up and back against the openings of the nostrils, allowing for optimal positioning and comfort, while not allowing breathing through the mouth. As each patient has a different shaped head, nose and mouth, this airway pressure mask system allows for multiple adjustments to optimize the flow of air while keeping the mouth closed.

This system allows for the use of alternative masks which have nasal pillows. For instance, a Resmed Airfit P30i or an InnoMed Technologies mask can be used. There are several more nasal pillow masks, such as a Swift LT, that have other air flow designs. Also, there are nasal masks and full masks that would benefit from the system being used here. The crown of the patient's head provides resistance for any nasal cushion movement and, independently, any adverse chin movement. This unique independent resistance design assures that any adverse movement of the nasal cushion will not adversely affect the mouth leakage and vice versa.

Basically, any mask having a nose and/or mouth section sides, and a forehead section or strap in one integral piece can be used. In another embodiment, any mask having just a nose and/or mouth section can also use this system of attachment.

In one embodiment, the crown strap section has a front crown strap 73 intersecting with a vertical head strap 74 which in turn intersects with the back head strap or neck strap 75. There are numerous possibilities of head straps and neck straps that can be used for the crown strap section

The PAP system described supra maximized comfort and fit. By having multiple points of unlimited adjustments thanks to the use of the hook and loop system, the device can fit many differently shaped and sized faces.

It should be noted that some people with sleep apnea sleep solely on their back and are “mouth breathers.” In such cases, in one embodiment, there is no need for a connection between the headgear and the mask.

In yet another embodiment, the mask may include just a mouth and/or nose section. In such an embodiment, a hook and loop strap 400 is connected at a first end to a loop or attachment device 401 which attached to the nose mask 402, with the second end of the loop strap attached to the first headgear connector strip 6. The nose mask is comprised of a body 404 of the nose mask which fits over the nostrils. More specifically, a hose nozzle 405 is positioned at the front of the body 404 of the nose mask 402. There is also a second loop or attachment device 408 on a second side of the nose mask 402 such that the second nose mask hook and loop strap 409 is looped through the second buckle 408 and attached through the second buckle on said second end of the said second side headgear connector strip 7. A similar arrangement exists for the full face masks 500, 501 except that there are can be up to two sets of Velcro straps 502, 503, and 504, 505 and 506, 507, and 508, 509 on each side of the full masks 500, 501.

Use of the PAP System

If a person has sleep apnea and is to use this device, the first step is to install and adjust the mouth piece. The mouthpiece should be cleaned before using.

To prepare to wear the headgear, it is helpful assemble the crown portion (larger piece) with the chin portion (smaller piece). Both hook and loop straps' lengths after the buckles should be approximately equal.

The headgear lay out on a flat surface with the ¾″ wide black hook and loop down, the crown portion farther from the user and the chin portion closer. The hook and loop strip nearest to the user's right hand is disconnected from the crown portion.

The headgear is raised up to the head, with the hook and loop to the front, and the crown portion is placed on it front to back on the crown of the head. The hook and loop strips are reconnected. While holding the strip in the right hand, disconnect the left strip and make the lengths after the buckles approximately equal.

The hook and loop strip(s) are tightened very gently so the headgear is not squeezing the head when the teeth are together. At this time, you should be able to relax the chin and the chin freely drop, leaving approximately ⅛″ to ¼″ gap between the teeth. It should be noted that in one embodiment, the headgear does not force the teeth to be clinched but be sufficiently loose to permit the chin to freely drop, thereby permitting a gap between your teeth through which you can talk and breathe. Also the soft loop side of the hook and loop will partially cover the ear. Since the headgear is not squeezing the head, there will be no ear discomfort. In fact, it is doubtful if it will be noticed at all.

There should be a theoretical straight line could be drawn from the center of the crown portion to the center of the chin portion. If the crown portion is too far forward, the headgear may slide forward when the head is on the pillow and mask leakage may occur.

When removing the headgear the right side Hook and loop strap should be removed only, with no need to remove the other straps.

The top side hook and loop straps of the mask and the top Hook and loop strap 39 of the headgear are positioned. With the mask in the left hand, the top hook and loop connector is slipped on to the one end of the mask. The swivel hose connector is then connected.

The left connector bridge and the right connector bridges are attached to the airway passage tube of the mask. This is first accomplished by positioning the left connector bridge on the inside of the left side of the air tubes of mask and slipping the narrow end tab through the plastic loop and connecting the loop to its first end of the strap and wrapping the first end second bottom strap around the left side of the air tubes of the mask and connecting it. This is repeated on the right side.

The nose pillow 200 is then installed by matching up the black arrows on the bottom part of the mask. The hook and loop connection at the crown of the head is then secured.

When properly located, the nose pillow 200 to which the two nostril pieces 202, 203 are attached should be located near the center of the nose. The nose pillow 200 is located near or against the nose. Gently hold the nose pillow 200 in place with the left hand and with the right hand jiggle the end of the nose for about 5-10 seconds. Next gentle jiggle the nose pillow 200 for 5 to 10 seconds.

While gently maintaining contact with the left hand between the nose and the nose pillow 200, with the right hand, the left connector bridge is connected to the left connector strap positioned on the headgear. This process is repeated on the right side while holding the cushion in place. The pressure on both sides of the face should feel equal and if not the process should be repeated.

The hook and loop on the side of the connectors are secured to the headgear. Apply very little tension.

To test the seal, the hole 201 in the nose pillow 200 is covered with a left forefinger. The hose attachment 46 should be covered with the right forefinger and with a thumb. The mouth is pressurized/depressurized to listen for air leaks. If an air leak is heard, both of the 1.5″ Hook and loop side connectors are disconnected and lifted toward the crown to add a little more sealing force on the nose. The system is then retested. If leaking still occurs, the nose pillow may need to be replaced.

This an example of one method of attaching one mask. Other methods can be used using this system, and a variety of masks can be used using this method of attachment. The example is not limiting.

While various embodiments of the present disclosure have been described above, it should be understood that they have been presented by way of example only, and not limitation. It will be apparent to persons skilled in the relevant art that various changes in form and detail can be made therein without departing from the spirit and scope of the disclosure. Thus, the breadth and scope of the present disclosure should not be limited by any of the above-described exemplary embodiments, but should be defined only in accordance with the following claims and their equivalents.

1) A positive airway pressure mask system, comprising:
a) a flexible, non-stretchable headgear, said headgear comprising:
i) a head crown section;
ii) a first side headgear connector strip comprising a first side strip of hook and loop material, a first end of said first side headgear connector strip in communication with a first side of said head crown section; and
iii) a second side headgear connector strip comprising a second side strip of hook and loop material, a first end of said second side headgear connector strip connecting to a second side of said head crown section;
iv) a chin strap, a first side of said chin strap connected to said first side headgear connector strip and a second side of said chin strap connected to said second side headgear connector strip:
v) at least one first side hook and loop strap, a first end of which is in communication with or integral with said first side headgear connector strip;
vi) at least one second side hook and loop strap, a first end of which is in communication with or integral with said second side head gear connector strip;
b) an air passage way tube comprising an air flow exit for directing air into at least one of a mouth or a set of nostrils, said air flow exit positioned between said first side section of said mask and a second side section of said mask;
said air passageway tube having the capability to align with and attach to both said hook and loop material of said first side headgear connector strip and said hook and loop strap of said second side headgear connector strip.

2) (canceled)

3) (canceled)

4) The positive airway pressure mask system of claim 1, wherein said head crown section further comprises a second crown strap.

5) The positive airway pressure mask system of claim 1, wherein said head crown section further comprises a vertical head strap intersecting at one end with said first crown strap and intersecting at said other end with a back head strap.

6) The positive airway pressure mask system of claim 1, further comprising:
a) a first buckle positioned at a second end of said first side headgear connector strip; and
b) a second buckle positioned at a second end of said second side headgear connector strip.

7) The positive airway pressure mask system of claim 6, wherein said chin strap comprises:
a) a chin rest;
b) a first hook and loop connection strap, a first end of said first hook and loop connection strap attached to an underside of the chin strap with the second end of said first hook and loop connection strap fitted through the first buckle;
c) a second hook and loop connection strap, a first end of said second hook and loop connection strap attached to an underside of the chin strap with the second end of said second hook and loop connection strap fitted with the second buckle.

8) The positive airway pressure mask system of claim 1, wherein said airflow exit is a nose pillow, said nose pillow comprising two nostril pieces and an exit hole.

9) A positive airway pressure mask system, comprising:
a) a non-stretchable headgear, said headgear comprising:
i) a head crown section;
ii) first side headgear connector strip comprising a first side strip of hook and loop material a first end of said first side headgear connector strip in communication with a first side of said head crown section;
iii) a second side headgear connector strip comprising a second side strip of hook and loop material, a first end of said second side headgear connector strip connecting to a second side of said head crown section;
iv) a chin strap, said chin strap connected to a second end of said first side headgear connector strip and to a second end of said second side headgear connector strip:
b) a nose mask, said nose mask comprising:
i) a body of the nose mask which fits over the nostrils;
ii) a hose nozzle positioned at a front of the body of the nose mask;
iii) a first hook and loop strap having a first end and a second end, said first end of said first hook and loop strap removably attached to a first side of said nose mask, said second end of said first hook and loop strap removably attached to said first side headgear connector strip; and
iv) a second hook and loop strap having a first end and second end, said first end of said first hook and loop strap removably attached to said second side of said nose mask, second end of said of said second hook and loop strap removably attached to said second headgear connector strip;
v) an air flow exit for directing air into a set of nostrils, said air flow exit positioned between said first side section of said mask and a second side section of said mask.

10) A positive airway pressure mask system, comprising:
a) a non-stretchable headgear, said headgear comprising:
i) a head crown section;
ii) a first side headgear connector strip comprising a first side strip of hook and loop material, a first end of said first side headgear connector strip in communication with a first side of said head crown section;
iii) a first side hook and loop strap having a first end, and a second end, said first end of said first hook and loop strap removably attached to said first side headgear connector strip;
iv) a second side headgear connector strip comprising a second side strip of hook and loop material, a first end of said second side headgear connector strip connecting to a second side of said head crown section;
v) a second hook and loop strap having a first end, and a second end, said first end of said first hook and loop strap removably attached to said second side headgear connector strip;
vi) a chin strap, said chin strap connected to a second end of said first side headgear connector strip and to a second end of said second side headgear connector strip:
b) a full mask, said full mask comprising:
i) a body of the full mask which fits over the mouth and nose;
ii) a hose nozzle positioned at a front of the body of the full mask;
iii) an air flow exit for directing air into the mouth, said air flow exit positioned between said first side section of said mask and a second side section of said mask;
wherein said second end of said first side hook and loop strap is connected to a first side of said full mask and said second end of at least one second side hook and loop strap is connected to the second side of said full mask.

11) The positive airway system of claim 10, further comprising:
a) an additional first side hook and loop strap connecting said first side of said full mask with said first side headgear connector strip; and
b) an additional second side hook and loop strap connecting said second side of said full mask with said second side headgear connector strip.

12) The positive airway system of claim 1, wherein:
a) said head crown section of said headgear further comprises an upwardly facing hook and loop strap;
b) said mask further comprising a forehead section, said forehead section comprising hook and loop strip attaching said air passage way tube to said head crown section of said headgear;
wherein said hook and loop strip is positioned on an underside of the forehead section and has the capability to align with said hook and loop strap of said first crown strap.

13) The positive airway pressure mask system of claim 12, further comprising;
a) a hose attachment positioned in the forehead section of said mask, wherein there are first and second crown hook and loop straps positioned on either side of said hose attachment, with a third material connecting the first crown hook and loop strap with the second crown hook and loop strap;
b) a first air passageway tube integral with said hose attachment, comprising a first side section of the mask to which a mask first side section hook and loop strap is attached; and
c) a second air passageway tube integral with said hose attachment, comprising a second side section of the mask to which a mask second side section hook and loop strap is attached.
